(19) United States
(12) Patent Application Publication
(10) Pub. No.: US 2016/0354126 A1
(43) Pub. Date: Dec. 8, 2016

(54) SURGICAL INSTRUMENT AND METHOD

(71) Applicant: Warsaw Orthopedic, Inc., Warsaw, IN (US)

(72) Inventors: Jerome Nayet, St Genis Pouilly (FR); Loic Josse, Yens Vaud (CH); Richard Assaker, Kain (BE)

(21) Appl. No.: 14/728,370

(22) Filed: Jun. 2, 2015

(51) Int. Cl. A61B 17/80; A61B 17/70

(52) U.S. Cl. CPC A61B 17/808; A61B 17/7074; A61B 17/7059

(57) ABSTRACT

A surgical instrument includes a member. A first arm extends from the member and includes a capture element. A second arm extends from the member and includes a capture element. The capture elements are engageable with a spinal plate. The arms define a perimeter of a passageway. Systems, implants and methods are disclosed.

### TECHNICAL FIELD

The present disclosure generally relates to medical devices for the treatment of musculoskeletal disorders, and more particularly to a surgical system for implant delivery to a surgical site and a method for treating a spine.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. As part of these surgical treatments, implants such as bone fasteners, plates, connectors and vertebral rods are often used to provide stability to a treated region. These implants can redirect stresses away from a damaged or defective region while healing takes place to restore proper alignment and generally support the vertebral members. For example, plates may be attached via the fasteners to the exterior of one or more vertebral members. This disclosure describes an improvement over these technologies.

### SUMMARY

In one embodiment, a surgical instrument is provided. The surgical instrument comprises a member. A first arm extends from the member and includes a capture element. A second arm extends from the member and includes a capture element. The capture elements are engageable with a spinal plate. The arms define a perimeter of a passageway. In some embodiments, systems, implants and methods are provided.

### BRIEF DESCRIPTION OF THE DRAWINGS

The present disclosure will become more readily apparent from the specific description accompanied by the following drawings, in which:

FIG. 1 is a perspective view of components of one embodiment of a surgical system in accordance with the principles of the present disclosure;

FIG. 2 is a plan view of the components shown in FIG. 1;

FIG. 3 is a break away view of the components shown in FIG. 1;

FIG. 4 is a plan view of the components shown in FIG. 1;

FIG. 5 is an enlarged, break away, side view of the components shown in FIG. 1;

FIG. 6 is a plan view of components of one embodiment of a surgical system in accordance with the principles of the present disclosure;

FIG. 7 is a plan view of components of one embodiment of a surgical system in accordance with the principles of the present disclosure;

FIG. 8 is a break away view of components of one embodiment of a surgical system in accordance with the principles of the present disclosure;

FIG. 9 is a break away view of components of one embodiment of a surgical system in accordance with the principles of the present disclosure;

FIG. 10 is a perspective view of components of one embodiment of a surgical system in accordance with the principles of the present disclosure;

FIG. 11 is a perspective view of components of one embodiment of a surgical system in accordance with the principles of the present disclosure; and

FIG. 12 is a perspective view of components of one embodiment of a surgical system in accordance with the principles of the present disclosure disposed with vertebrae.

### DETAILED DESCRIPTION

The exemplary embodiments of the surgical system and related methods of use disclosed are discussed in terms of medical devices for the treatment of musculoskeletal disorders and more particularly, in terms of a surgical system for implant delivery to a surgical site and a method for treating a spine.

In some embodiments, the surgical system includes a surgical instrument, such as, for example, a cervical plate holder. In some embodiments, the surgical instrument is configured to facilitate stability when attached to an implant. In some embodiments, the surgical instrument includes monolithic components configured to facilitate cleaning. In some embodiments, the surgical instrument is configured to improve and adapt to a selected surgical procedure. In some embodiments, the surgical instrument is configured to be freely positioned and/or translated relative to a spinal implant, such as, for example, a cervical plate for attachment therewith. In some embodiments, the surgical instrument is configured to be attached with the cervical plate and freely positionable and/or translatable relative to the cervical plate. In some embodiments, the surgical instrument is configured to be freely angulated and/or rotated relative to a spinal implant, such as, for example, a cervical plate for attachment therewith. In some embodiments, the surgical instrument is configured to be attached with the cervical plate and freely angulated and/or rotated relative to the cervical plate to facilitate visibility. In some embodiments, the distal end of the surgical instrument includes teeth and/or ridges disposed on an inner surface thereof to facilitate mating and/or gripping with the plate, while allowing rotation and/or translation to facilitate attachment with the plate and/or visibility of surgical system components.

In some embodiments, the surgical system includes a surgical instrument having a single part implant holder, such as, for example, a spinal plate holder. In some embodiments, the surgical instrument comprises a sturdy configuration to facilitate manipulation. In some embodiments, the surgical instrument includes a passageway, such as, for example, a window to facilitate visibility of surgical system components, tissue and/or a surgical site. In some embodiments, the window is configured to facilitate insertion of other surgical instruments. In some embodiments, the surgical instrument comprises metal. In some embodiments, the surgical instrument comprises plastic. In some embodiments, the surgical instrument is configured for a single use and/or disposable.

In some embodiments, the surgical system includes a plate holder, a plate and a screw driver/awl. In some embodiments, the surgical instrument can be easily connected and/or disconnected from a spinal implant, such as, for example, a cervical plate upon fixation of the cervical plate with vertebrae. In some embodiments, the surgical instrument includes a window for visibility and access with other instruments such as a screw driver. In some embodiments, the window is configured to facilitate insertion of other surgical instruments.

In one embodiment, one or all of the components of the surgical system are disposable, peel-pack, pre-packed sterile devices used with an implant. One or all of the components of the surgical system may be reusable. The surgical system may be configured as a kit with multiple sized and configured components. In one embodiment, the surgical system includes one or a plurality of guides, each guide be configured for disposal with a plate at a different angle.

In some embodiments, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed surgical system and methods may be alternatively employed in a surgical treatment with a patient in a prone or supine position, and/or employ various surgical approaches to the spine, including anterior, posterior, posterior mid-line, direct lateral, postero-lateral, and/or antero-lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

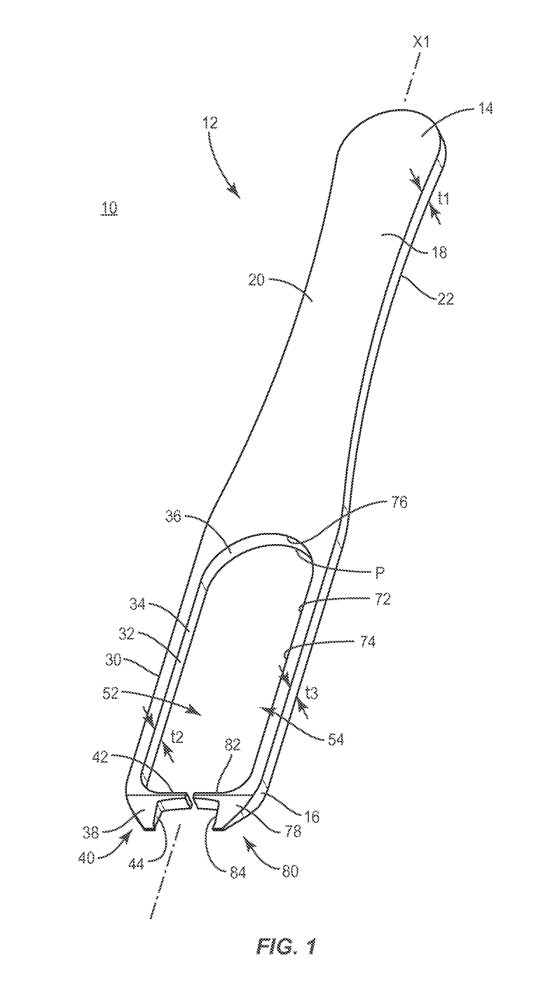

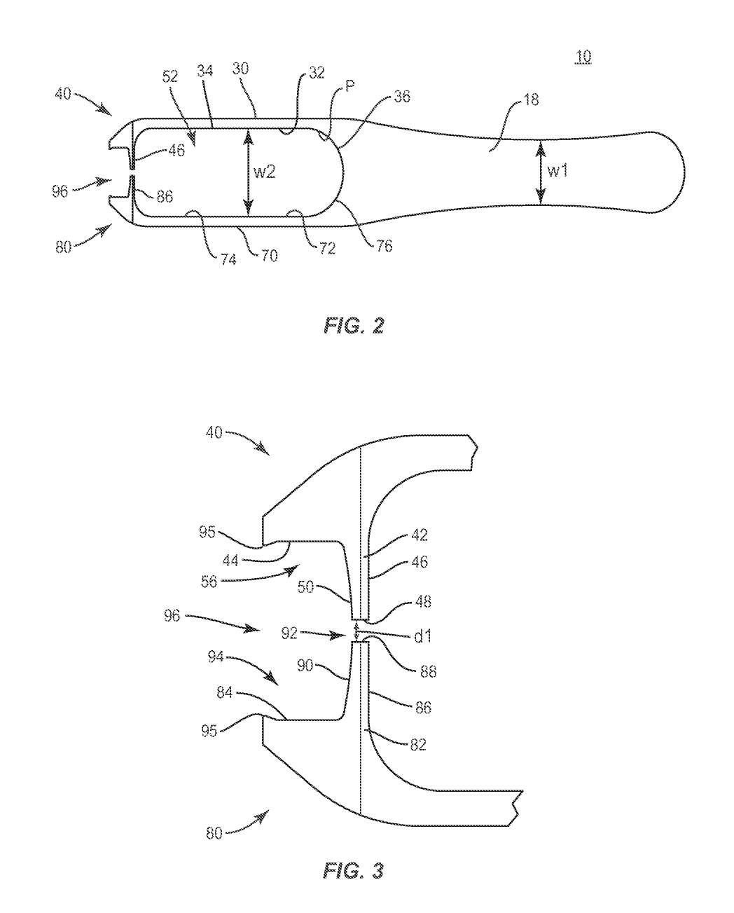

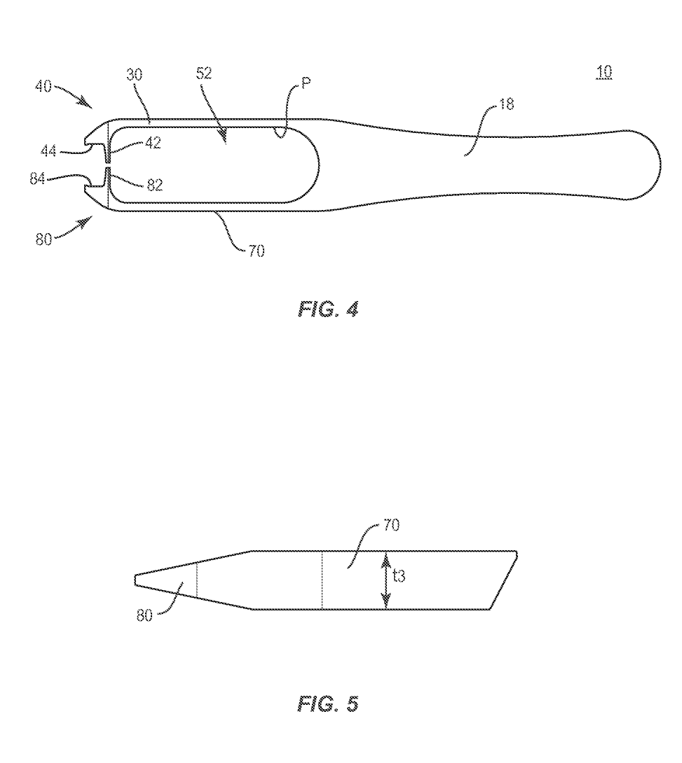

The following discussion includes a description of a surgical system and related methods of employing the surgical system in accordance with the principles of the present disclosure. Alternate embodiments are also disclosed. Reference is made in detail to the exemplary embodiments of the present disclosure, which are illustrated in the accompanying figures. Turning to FIGS. 1-5, there are illustrated components of a surgical system 10, including a surgical instrument, such as, for example, a plate holder 12.

The components of surgical system 10 can be fabricated from biologically acceptable materials suitable for medical applications, including metals, synthetic polymers, ceramics and bone material and/or their composites. For example, the components of surgical system 10, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL®), ceramics and composites thereof such as calcium phosphate (e.g., SKELITE™) thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of surgical system 10 may have material composites, including the above materials, to achieve various desired characteristics such as strength, rigidity, elasticity, compliance, biomechanical performance, durability and radiolucency or imaging preference. The components of surgical system 10, individually or collectively, may also be fabricated from a heterogeneous material such as a combination of two or more of the above-described materials. The components of surgical system 10 may be monolithically formed, integrally connected or include fastening elements and/or instruments, as described herein.

The components of surgical system 10 including plate holder 12 are employed, for example, with a minimally invasive procedure, including percutaneous techniques, mini-open and open surgical techniques to deliver and introduce an implant, such as, for example, one or a plurality of bone fasteners and/or spinal plates, at a surgical site within a body of a patient, for example, a section of a spine. In some embodiments, plate holder 12 delivers and introduces a spinal plate for fixation with an anterior side of a spine. In some embodiments, plate holder 12 is configured to facilitate drilling and/or tapping of tissue, such as, for example, vertebrae, and/or guiding, fastening and/or driving fasteners, such as, for example, bone screws with the vertebrae using one or a plurality of surgical tools, which tools may be alternately disposed with plate holder 12 during a procedure.

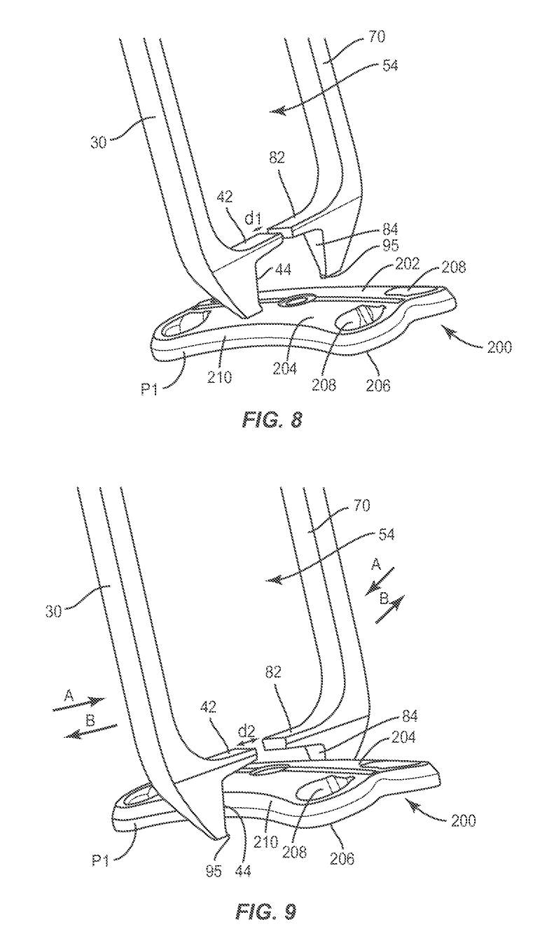

Plate holder 12 is configured for connecting to an implant, such as, for example, a cervical plate 200 (FIG. 8), as described herein. Plate holder 12 extends along a longitudinal axis X1 between an end 14 and an end 16. Plate holder 12 includes a member, such as, for example a handle 18. Handle 18 includes a planar surface 20 and a planar surface 22. Handle 18 includes a uniform thickness t1 that extends between surfaces 20, 22. Handle 18 includes a width w1. In some embodiments, width w1 has an hourglass shape, as shown in FIG. 1. In some embodiments, width w1 is aligned with a width w2 of a passageway, which includes a window of a plate holder. In some embodiments, thickness t1 may have various configurations, such as, for example, undulating, irregular, non-uniform, variable and/or tapered. In some embodiments, width w1 may have various configurations, such as, for example, undulating, irregular, non-uniform, variable and/or tapered.

In some embodiments, handle 18 is substantially flat. In some embodiments, handle 18 may have various cross-section configurations, such as, for example, cylindrical, oval, oblong, triangular, rectangular, square, polygonal, irregular, uniform, non-uniform, variable and/or tapered. In some embodiments, handle 18 includes a gripping surface configured to facilitate maneuvering of plate holder 12. In some embodiments, all or only a portion of the gripping surface may have alternate surface configurations, such as, for example, rough, threaded for connection with surgical instruments, arcuate, undulating, porous, semi-porous, dimpled, polished and/or textured according to facilitate gripping.

An arm 30 extends from handle 18. In one embodiment, arm 30 is monolithically formed with handle 18. In some embodiments, arm 30 is integrally connected with handle 18 or includes fastening elements and/or instruments. Arm 30 extends between surfaces 20, 22 and includes a uniform thickness t2. In some embodiments, thickness t2 is equal to thickness t1. In some embodiments, thickness t2 may have various configurations, such as, for example, undulating, irregular, non-uniform, variable and/or tapered. In some embodiments, arm 30 extends in a cantilever configuration from handle 18. In some embodiments, the material of handle 18 and/or arm 30 is a pliable, flexible, spring-like material and/or a material configured to retain its configuration, such as, for example, Nitinol. In some embodiments, arm 30 is inwardly biased to a contracted orientation, as described herein.

Arm 30 includes a surface 32. In some embodiments, surface 32 includes a linear portion 34 and a concave portion 36. In some embodiments, surface 32 may have alternate configurations, such as, for example, arcuate, irregular, uniform, non-uniform, variable and/or tapered.

Arm 30 includes a surface 38 that defines a capture element 40. Capture element 40 extends from arm 30 in a tapered configuration. In some embodiments, capture element 40 may extend from arm 30 in alternate configurations, such as, for example, arcuate, irregular, uniform, non-uniform and/or variable. Capture element 40 includes a flange 42 and an engagement surface 44. Flange 42 includes surfaces 46, 48, 50, as shown in FIG. 3. Surfaces 32, 46 define a portion of a perimeter P that defines a passageway 52, as described herein.

Surfaces 44, 50 define cavity 56 configured for disposal of a portion of plate 200, as described herein. In some embodiments, all or only a portion of surfaces 44 and/or 50 may have alternate surface configurations, such as, for example, planar, rough, undulating, porous, semi-porous, dimpled, polished and/or textured to facilitate capture of plate 200.

An arm 70 extends from handle 18 in a spaced apart relation to arm 30. In one embodiment, arm 70 is monolithically formed with handle 18. In some embodiments, arm 70 is integrally connected with handle 18 or includes fastening elements and/or instruments. Arm 70 extends between surfaces 20, 22 and includes a thickness t3. In some embodiments, thickness t3 is equal to thickness t1 and/or thickness t2. In some embodiments, thickness' t1 t2, t3 have a uniform dimension between surfaces 20, 22. In some embodiments, thickness' t1, t2 and/or t3 may be uniformly increasing or decreasing between surfaces 20, 22. In some embodiments, thickness 13 may have various configurations, such as, for example, undulating, irregular, non-uniform, variable and/or tapered. In some embodiments, arm 70 extends in a cantilever configuration from handle 18. In some embodiments, the material of arm 70 is a pliable, flexible, spring-like material and/or a material configured to retain its configuration, such as, for example, Nitinol. In some embodiments, arm 70 is inwardly biased to a contracted orientation, as described herein.

Arm 70 includes a surface 72. In some embodiments, surface 72 includes a linear portion 74 and a concave portion 76. Concave portion 76 is connected with concave portion 36. In some embodiments, surface 72 may have alternate configurations, such as, for example, arcuate, irregular, uniform, non-uniform, variable and/or tapered.

Arm 70 includes a surface 78 that defines a capture element 80. Capture element 80 extends from arm 70 in a tapered configuration. In some embodiments, capture element 80 may extend from arm 70 in alternate configurations, such as, for example, arcuate, irregular, uniform, non-uniform and/or variable. Capture element 80 includes a flange 82 and an engagement surface 84. Flange 82 includes surfaces 86, 88, 90, as shown in FIG. 3. Surface 72, 86 define a portion of perimeter P that defines passageway 52, as described herein.

Surfaces 84, 90 define a cavity 94 configured for disposal of a portion of plate 200, as described herein. In some embodiments, all or only a portion of surfaces 84, 90 may have alternate surface configurations, such as, for example, planar, rough, undulating, porous, semi-porous, dimpled, polished and/or textured to facilitate capture of plate 200. Cavity 94 communicates with cavity 56 to define capture cavity 96 configured for disposal of plate 200. In some embodiments, plate holder 12 may include one or a plurality of capture cavities, which may be in communication and/or spaced apart.

Surfaces 48, 88 are spaced apart to define a slot 92. Slot 92 communicates with and connects passageway 52 and cavity 96, as described herein. Slot 92 is configured to facilitate expansion of arms 30, 70, as described herein, in a range of movable expansion and contraction of arms 30, 70, as described herein. In some embodiments, slot 92 is configured to provide a limit of deformation of arms 30, 70 in compression. In some embodiments, this deformation limit resists and/or prevents permanent deformation of arms 30, 70 to facilitate connection with plate 200,

Surgical system 10 includes plate 200, as shown in FIGS. 8 and 9. Plate 200 includes a stratum 202 having a surface 204 and a surface 206. Surface 206 is configured to engage tissue, such as, for example, an anterior portion of vertebrae. Stratum 202 defines a series of openings 208 extending between surface 204 and surface 206. Openings 208 are configured for disposal of permanent implantable fixation elements, such as, for example, bone screws for attaching plate 200 with tissue. Stratum 202 defines an edge 210 disposed around an entire periphery P1 of plate 200. In some embodiments, edge 210 includes a surface angle, such as, for example, a chamfer configured to facilitate connection of plate 200 with plate holder 12.

Arms 30, 70 are resiliently configured for disposal between a contracted orientation, as shown in FIG. 8, such that arms 30, 70 are inwardly biased, in the direction shown by arrows A, relative to handle 18 and an expanded orientation, as shown in FIG. 9, such that arms 30, 70 are rotatable and/or pivotable, in the direction shown by arrows B, about handle 18 and a spinal implant, such as, for example, plate 200 is disposable with cavity 96. In the contracted orientation, capture elements 40, 80 are relatively disposed such that surfaces 48, 88 are spaced apart a distance d1. In the expanded orientation, capture elements 40, 80 are relatively disposed such that surfaces 48, 88 are spaced apart a distance d2 and oriented to receive plate 200.

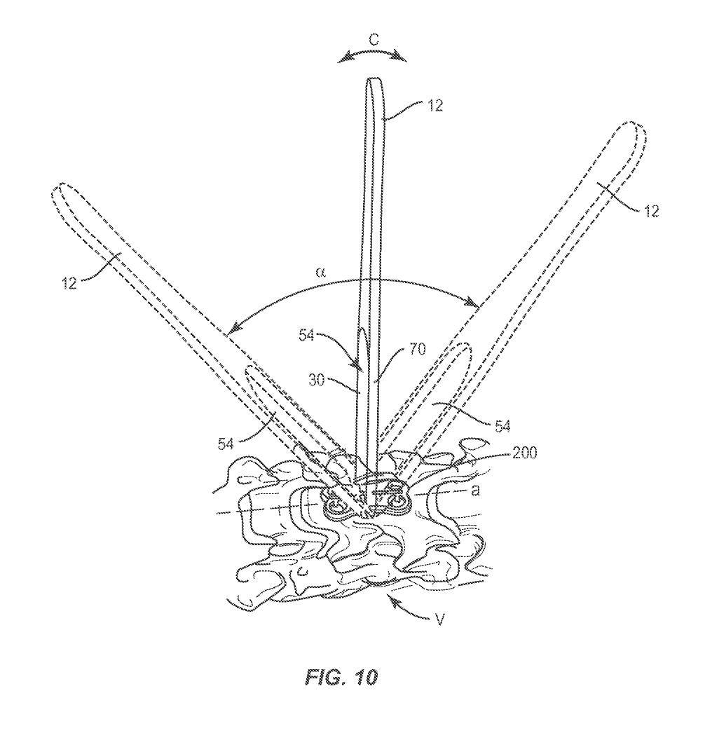

In some embodiments, in the expanded orientation, plate 200 is disposed adjacent capture elements 40, 80 such that arms 30, 70 are biased into engagement with plate 200 and capture elements 40, 80 engage plate 200 in a friction fit and/or pressure fit. In some embodiments, plate holder 12 is selectively angulated and/or rotated relative to plate 200, in the direction shown by arrows C in FIG. 10, for disposal in an orientation for attachment with plate 200. In some embodiments, plate holder 12 is attached with plate 200 and rotated relative to plate 200, in the direction shown by arrows C in FIG. 10, while attached with plate 200 to facilitate orientation of a window 54 of passageway 52, as described herein, and visibility for viewing components of surgical system 10 and/or a surgical site, and/or insertion/delivery of components of surgical system 10 and/or one or more surgical tools, as well as guiding fasteners to penetrate tissue. Arms 30, 70 are disposable at an angular orientation a relative to an axis a of plate 200 to facilitate orientation of window 54, as described herein. In some embodiments, angular orientation a is in a range of +/−0 through 60 degrees to facilitate orientation of window 54, as described herein. In some embodiments, angular orientation a is in a range of +/− through 70 degrees to facilitate orientation of window 54, as described herein.

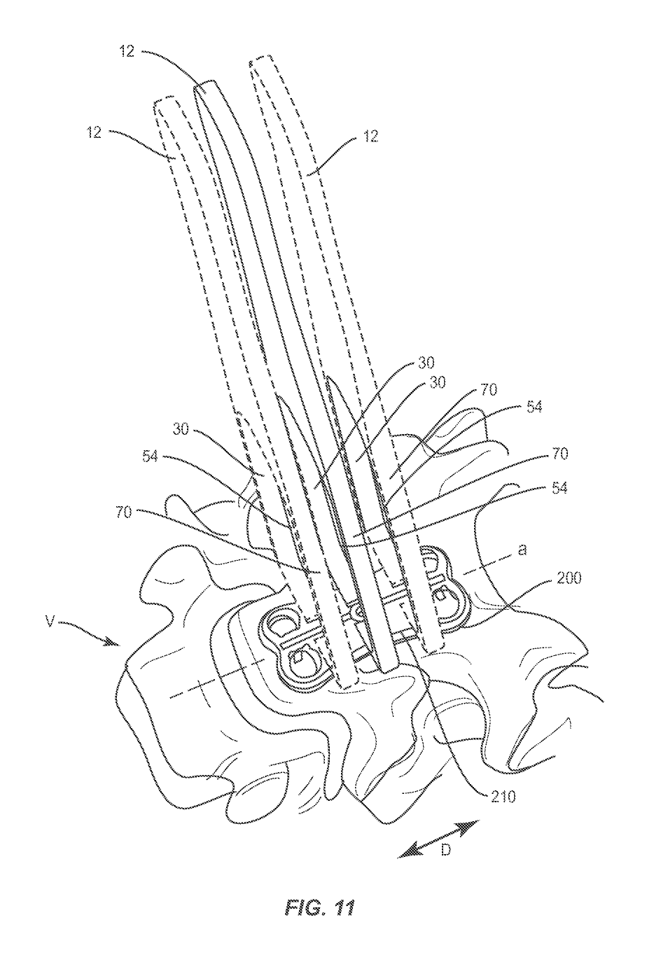

In some embodiments, plate holder 12 is selectively positionable and/or translatable relative to plate 200, in the direction shown by arrows D in FIG. 11, for disposal in an orientation for attachment with plate 200. Arms 30, 70 are disposable at angular orientation a relative to an axis a of plate 200 at various positions along plate 200 in the direction shown by arrows D to facilitate orientation of window 54. In some embodiments, plate holder 12 is attached with plate 200 and translatable relative to plate 200, in the direction shown by arrows D in FIG. 11, while attached with plate 200 to facilitate orientation of window 54 and visibility for viewing components of surgical system 10 and/or a surgical site, and/or insertion/delivery of components of surgical system 10 and/or one or more surgical tools, as well as guiding fasteners to penetrate tissue. In some embodiments, as shown in FIG. 3, end 16 includes teeth and/or ridges 95 to facilitate mating and/or gripping plate 200, while allowing rotation and/or translation relative to plate 200, as described herein, to facilitate attachment with plate 200 and/or visibility of surgical system 10 components.

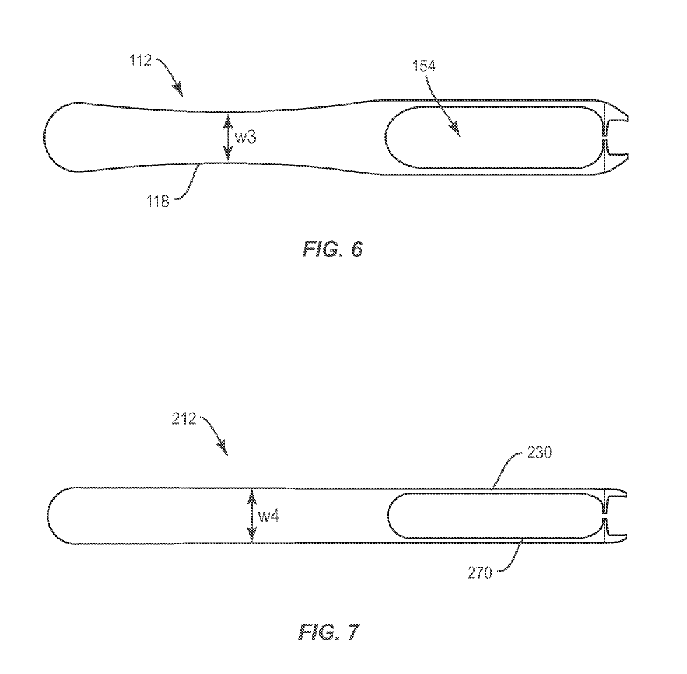

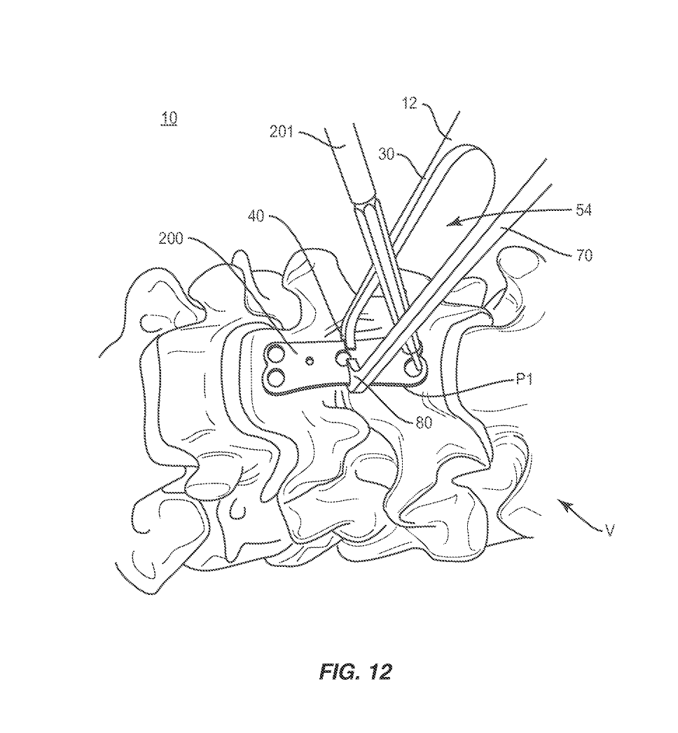

Window 54 extends width w2 and is configured to facilitate visibility for viewing components of surgical system 10 and/or a surgical site, and/or insertion/delivery of components of surgical system 10 and/or one or more surgical tools, as well as guiding fasteners to penetrate tissue. Window 54 is configured to guide at least one surgical tool, such as, for example, a drill 201 in alignment with plate 200, as shown in FIG. 12 and described herein. In some embodiments, the at least one surgical tool includes a plurality of tools that can be alternately disposed with window 54, as described herein. In some embodiments, window 54 may have alternate configurations, such as, for example, oval, oblong, triangular, rectangular, square, polygonal, irregular, uniform, non-uniform, variable and/or tapered. In one embodiment, as shown in FIG. 6, a plate holder 112, similar to plate holder 12 described herein, includes a reduced profile handle 118 having width w3 disposed in alignment with a window 154 having width w3. In one embodiment, as shown in FIG. 7, a plate holder 212, similar to plate holder 12 described herein, includes a uniform outer profile and width w4 along a substantial portion of a handle 218 and arms 230, 270.

In assembly, operation and use, surgical system 10, similar to the systems and methods described herein, is employed to treat a selected section of vertebrae V, as shown in FIGS. 10-12. A medical practitioner obtains access to a surgical site including vertebrae V in any appropriate manner, such as through incision and retraction of tissues. In some embodiments, surgical system 10 can be used in any existing surgical method or technique including open surgery, mini-open surgery, minimally invasive surgery and percutaneous surgical implantation, whereby vertebrae V is accessed through a mini-incision, or sleeve that provides a protected passageway to the area. Once access to the surgical site is obtained, the particular surgical procedure can be performed for treating the spine disorder. In some embodiments, one or all of the components of surgical system 10 can be delivered or implanted as a pre-assembled device or can be assembled in situ. The components of surgical system 10 may be completely or partially revised, removed or replaced.

An incision is made in the body of a patient and a cutting instrument (not shown) creates a surgical pathway for implantation of components of surgical system 10 with an anterior portion of vertebrae V. A preparation instrument (not shown) can be employed to prepare tissue surfaces of vertebrae V, as well as for aspiration and irrigation of a surgical region.

Plate holder 12, similar to that described herein, is connected with plate 200 and/or bone fasteners (not shown) for orientation and delivery of the components of surgical system 10 along the surgical pathway. Plate holder 12 introduces the components of surgical system 10 along the surgical pathway to implant plate 200 and/or bone fasteners in substantial alignment to attach plate 200 and/or bone fasteners with vertebrae V.

Plate holder 12 is disposed adjacent plate 200 such that arms 30, 70 are disposed in a contracted orientation, as described herein and shown in FIG. 8. Plate holder 12 is manipulated for engagement with plate 200. In some embodiments, plate holder 12 can be selectively angulated and/or rotated relative to plate 200, in the direction shown by arrows C in FIG, 10, for disposal in an orientation for attachment with plate 200, as described herein. In some embodiments, plate holder 12 is selectively positionable and/or translatable relative to plate 200, in the direction shown by arrows D in FIG. 11, for disposal in an orientation for attachment with plate 200, as described herein.

Arms 30, 70 engage plate 200 such that capture elements 40, 80 engage edge 210. Arms 30, 70 rotate, in the direction shown by arrows B in FIG. 9, about handle 18 to the expanded orientation, as described herein. Plate 200 is disposed with cavity 96. Ridges 95 engage periphery P1 to facilitate mating and/or gripping plate of plate 200 in a friction fit and/or pressure fit with plate holder 12.

Plate holder 12 is attached with plate 200 and handle 18 can be rotated and/or translated, as described herein, relative to plate 200 to facilitate orientation of window 54 for viewing components of surgical system 10, the surgical site, insertion/delivery of components of surgical system 10 and/or one or more surgical tools.

Surgical tool 201 is passed through window 54 to facilitate attachment of plate 200 with vertebrae V. Window 54 guides surgical tool 201 in alignment with plate 200. In some embodiments, plate holder 12 is an adaptable instrument configured to perform multiple applications during a surgical procedure. In some embodiments, plate holder 12 guides surgical tool 201, such as, for example, a drill, tap, driver and/or an awl, as well as guiding fasteners to penetrate tissue. For example, surgical tool 201 is passed through window 54. In some embodiments, surgical tool 201 comprises a drill or tap that is guided through window 54 and can create pilot holes or the Ike through openings 208 in selected vertebra of vertebrae V for receiving fixation elements, such as, for example, bone screws (not shown). In some embodiments, surgical tool 201 comprises a driver that is guided through window 54 to fasten the bone screws with openings 208 and vertebrae V to fasten plate 200 with vertebrae V via the bone screws and the surgical tools employed with plate holder 12. With plate 200 fastened with vertebrae V, plate holder 12 is disengaged from plate 200 such that ridges 95 release from periphery P1. Arms 30, 70 are biased to the contracted orientation.

Upon completion of the procedure, the surgical instruments, assemblies and non-implanted components of surgical system 10 are removed and the incision is closed. Surgical system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of surgical system 10. In some embodiments, surgical system 10 may include one or a plurality of plates, connectors and/or bone fasteners for use with a single vertebral level or a plurality of vertebral levels.

In some embodiments, surgical system 10 includes one or more fasteners, not shown, for attaching plate 200 with tissue, as described herein. In some embodiments, the fasteners may be engaged with tissue in various orientations, such as, for example, series, parallel, offset, staggered and/or alternate vertebral levels, In some embodiments, one or more of the fasteners may comprise multi-axial screws, sagittal angulation screws, pedicle screws, mono-axial screws, uni-planar screws, facet screws, fixed screws, tissue penetrating screws, conventional screws, expanding screws, wedges, anchors, buttons, clips, snaps, friction fittings, compressive fittings, expanding rivets, staples, nails, adhesives, posts, fixation plates and/or posts.

In one embodiment, surgical system 10 includes an agent, which may be disposed, packed, coated or layered within, on or about the components and/or surfaces of surgical system 10. In some embodiments, the agent may include bone growth promoting material, such as, for example, bone graft to enhance fixation of the components and/or surfaces of surgical system 10 with vertebrae. In some embodiments, the agent may include one or a plurality of therapeutic agents and/or pharmacological agents for release, including sustained release, to treat, for example, pain, inflammation and degeneration.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical instrument comprising:
a member;
a first arm extending from the member and including a capture element; and
a second arm extending from the member and including a capture element, the capture elements being engageable with a spinal plate, the arms defining a perimeter of a passageway.

2. A surgical instrument as recited in claim 1, wherein the member and the arms are monolithic.

3. A surgical instrument as recited in claim 1, wherein the passageway includes a window.

4. A surgical instrument as recited in claim 1, wherein the passageway includes a window and is configured to guide at least one surgical tool in alignment with the spinal plate.

5. A surgical instrument as recited in claim 4, wherein the at least one surgical tool comprises a plurality of tools that can be alternately disposed with the passageway.

6. A surgical instrument as recited in claim 1, wherein the arms are expandable such that the capture elements capture the spinal plate.

7. A surgical instrument as recited in claim 1, wherein the arms are biased such that the capture elements capture the spinal plate.

8. A surgical instrument as recited in claim 1, wherein the arms are disposable at an angular orientation relative to the spinal plate such that the capture elements capture the spinal plate, the angular orientation including an angle in a range of +/−0 through 60 degrees.

9. A surgical instrument as recited in claim 1, wherein the member and the arms include a unform thickness.

10. A surgical instrument as recited in claim 9, wherein the uniform thickness extends between a first planar surface and a second planar surface of the member and the arms.

11. A surgical instrument as recited in claim 1, wherein the member includes a width dimension aligned with the passageway.

12. A surgical instrument as recited in claim 1, wherein the perimeter includes a linear surface of the first arm and a flange of the capture element of the first arm, the flange being disposed in a perpendicular orientation relative to the linear surface.

13. A surgical instrument as recited in claim 1, wherein the perimeter includes a concave surface and a plurality of linear surfaces of the arms.

14. A surgical instrument as recited in claim 1, wherein the capture elements define at least one cavity configured for disposal of the spinal plate.

15. A surgical instrument as recited in claim 14, wherein the capture elements include flanges that define a slot disposed in communication with the cavity and the passageway.

16. A surgical instrument as recited in claim 1, wherein at least one of the capture elements extend from the arms in a tapered configuration.

17. A surgical instrument as recited in claim 1, wherein the capture elements engage the spinal plate in a friction fit.

18. A plate holder comprising:
a handle;
a first arm extending from the handle and including a capture element having a flange and an engagement surface; and
a second arm extending from the handle and including a capture element having a flange and an engagement surface, the arms being expandable such that the capture elements capture a spinal plate in a friction fit,
the arms and the flanges defining a perimeter of a passageway, the passageway including a window and is configured to guide at least one surgical tool in alignment with the spinal plate.

19. A spinal implant system comprising:
a spinal p ate;
at least one fastener configured to attach the spinal plate with tissue; and
a surgical instrument comprising
a member,
a first arm extending from the member and including a capture element, and
a second arm extending from the member and including a capture element, the capture elements being engageable with the spinal plate, the arms defining a perimeter of a passageway.

20. A spinal implant system as recited in claim 19, wherein the passageway includes a window and is configured to guide a surgical tool in alignment with the spinal plate and engage the at least one fastener.
